(19) United States
(12) Patent Application Publication
(10) Pub. No.: US 2008/0249035 A1
(43) Pub. Date: Oct. 9, 2008

(54) Polymorph of Clarithromycin (Form V)

(75) Inventor: Michael GRUSS, Aachen (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 12/060,370

(22) Filed: Apr. 1, 2008

(30) Foreign Application Priority Data
Apr. 3, 2007 (DE) 10 2007 016 367.5

(51) Int. Cl. A61K 31/7048; C07H 17/08; A61P 31/04

(52) U.S. Cl. 514/29; 536/7.2; 536/7.5

(57) ABSTRACT

A polymorphic form of clarithromycin (form V) which exhibits a characteristic X-ray diffraction pattern, a method for producing such polymorphic clarithromycin, and the use of such polymorphic clarithromycin to treat bacterial infections.

### BACKGROUND OF THE INVENTION

The invention relates to a polymorphic form of clarithromycin (form V), and to the production and use thereof.

Clarithromycin (6-O-methylerythromycin A) is a semisynthetic macrolide antibiotic, which displays excellent antibiotic efficacy against Gram-positive bacteria, some Gram-negative bacteria, anaerobic bacteria, mycoplasmas and Chlamydia. Clarithromycin is stable in acidic conditions and can be administered orally. It is particularly suitable for the treatment of infections of the upper respiratory tract in children and adults.

The following crystalline modifications of clarithromycin are already known: form I (WO 98/04573), form II (NAVSUY; G. A. Stephenson, et al.; J. Pharm. Sci. (1997), 86, 1239; EP-A 915 899) and form IV (WO 01/44262). An amorphous form, a hydrated form and an anhydrous form have also been described (WO 2005/61524). Furthermore, crystalline solvates of ethanol, isopropyl acetate, tetrahydrofuran and isopropanol (form 0, WO 98/31699), of acetonitrile (form III, WO 01/40242) and of methanol (WANNUU; H. Iwasaki et al., Acta Crystallographica, Section C: Crystal Structure Communications 1993, 49(6), 1227-30) are known. A hydrated hydrochloride is described in M. Parvez et al., Acta Crystallographica, Section C: Crystal Structure Communications 2000, 56(9), E398-9.

The polymorphic forms of clarithromycin known in the state of the art vary with respect to their thermodynamic stability and solubility, among other things.

On the one hand, high thermodynamic stability is usually associated with good stability in storage, but may have disadvantages in respect of bioavailability and other pharmacokinetic parameters. Thus, high thermodynamic stability usually correlates with relatively low solubility, which for its part exerts an influence on, among other things, the release behaviour and bioavailability of the active substance.

On the other hand, good solubility is usually associated with good bioavailability, but may have disadvantages with respect to stability in storage. Thus, good solubility usually correlates with relatively low thermodynamic stability, which for its part can have the result that, among other things, the polymorphic form is transformed to a thermodynamically more stable form during storage, which can, for example, alter the pharmacokinetic parameters.

Accordingly there is a need for crystalline modifications that represent a balanced compromise between maximum thermodynamic stability on the one hand and maximum solubility on the other hand.

### SUMMARY OF THE INVENTION

It is therefore an object of the invention to provide a crystalline form of clarithromycin that has advantages relative to the previously known polymorphic forms of clarithromycin.

Basically these advantages can be based on various properties of the crystalline form, for example its general properties, such as solubility, density, hygroscopicity; its thermodynamic properties, such as thermodynamic stability, melting point; its electrical properties, such as dielectric constant, conductivity; its mechanical properties, such as hardness, breaking strength, friability, elasticity; its optical properties, such as colour, transparency, refraction; and so on. These advantages can basically have effects in various respects, for example with respect to improved stability in storage, reactivity, pH-stability, release of the active substance, solubility in vivo, absorption, bioavailability, processability to suitable pharmaceutical forms, etc.

These and other objects of the invention are achieved by providing a new crystalline modification of clarithromycin which exhibits X-ray diffraction reflections at 5.64±0.20 2Θ, 6.84±0.20 2Θ, 8.92±0.20 2Θ, 11.58±0.20 2Θ, 12.99±0.20 2Θ and 16.73±0.20 2Θ.

It has been surprisingly found that under suitable conditions a crystalline modification could be crystallized from a ketone that was previously unknown and is thermodynamically less stable than the known form II.

WO 98/04574 discloses that treatment of clarithromycin with a ketone having 3 to 12 carbon atoms, for example acetone, methyl ethyl ketone, pentan-2-one or 3-pentan-3-one, leads to form II. According to example 1 in WO 98/04574, form II can be obtained by crystallization from acetone. Moreover, it is disclosed in WO 98/04573 that treatment of clarithromycin with solvent mixtures, which contain acetone among other things, leads to form I.

However, it was found, surprisingly, that under suitable conditions form V according to the invention can be obtained from ketones. Important factors appear to be, in particular, the concentration of clarithromycin in solution, the drying time and the cooling rate during crystallization. If the experimental conditions are not selected carefully, some other form, for example form I or form II, is produced.

The crystalline modification of clarithromycin according to the invention (form V) comprises X-ray diffraction reflections at 5.64±0.20 2Θ, 6.84±0.20 2Θ, 8.92±0.20 2Θ, 11.58±0.20 2Θ, 12.99±0.20 2Θ and 16.73±0.20 2Θ.

Preferably the crystalline modification according to the invention additionally comprises at least one X-ray diffraction reflection selected from the group consisting of 8.18±0.20 2Θ, 9.49±0.20 2Θ, 10.73±0.20 2Θ, 11.08±0.20 2Θ, 12.21±0.20 2Θ, 13.76±0.20 2Θ, 13.92±0.20 2Θ, 17.09±0.20 2Θ, 17.77±0.20 2Θ, 18.09±0.20 2Θ, 19.18±0.20 2Θ, 20.31±0.20 2Θ, 20.60±0.20 2Θ and 20.91±0.20 2Θ.

### BRIEF DESCRIPTION OF THE DRAWINGS

The invention will be described in further detail hereinafter with reference to the accompanying drawing figures, in which:

FIG. 1 shows an X-ray powder diffraction pattern of form V (middle) compared with form I (top) and form II (bottom).

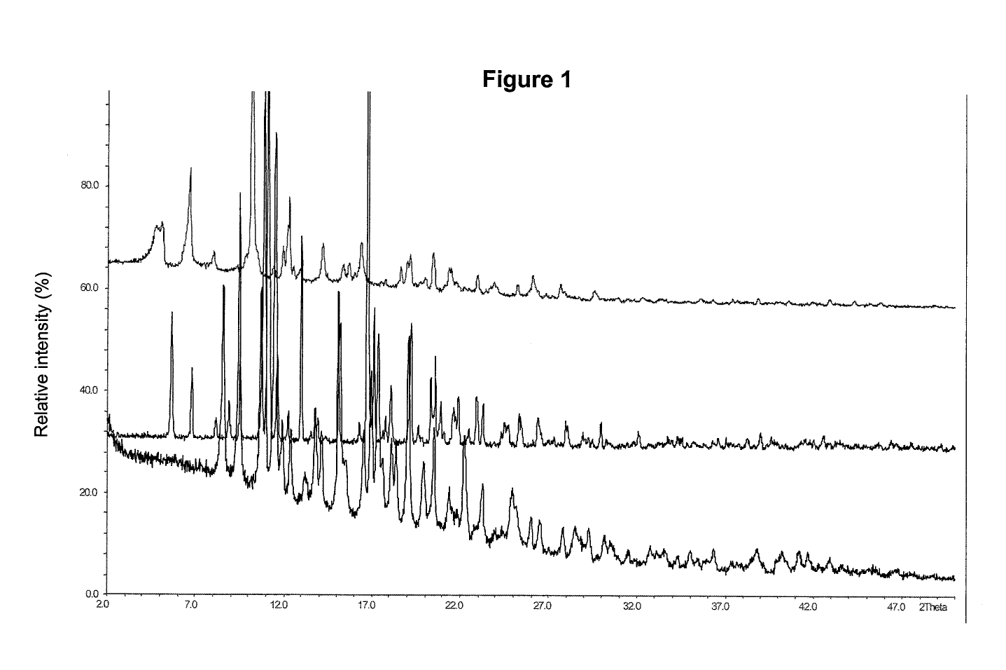

FIG. 2 shows a DSC recording of form V.

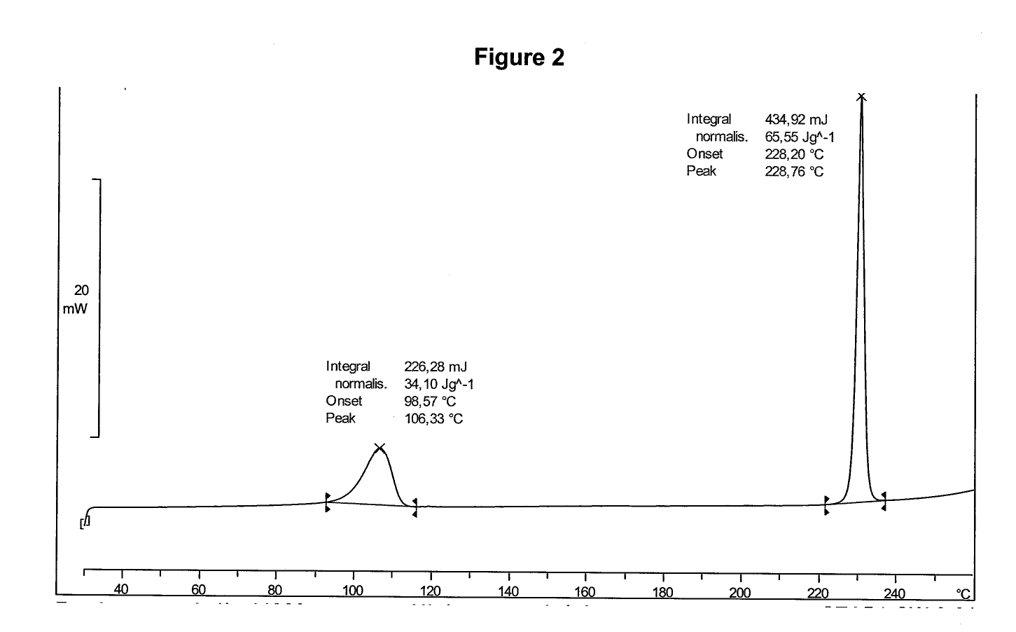

FIG. 3 shows an X-ray powder diffraction pattern with reflections of form V and form II (middle) compared with phase-pure diffraction patterns of form II (top) and form V (bottom).

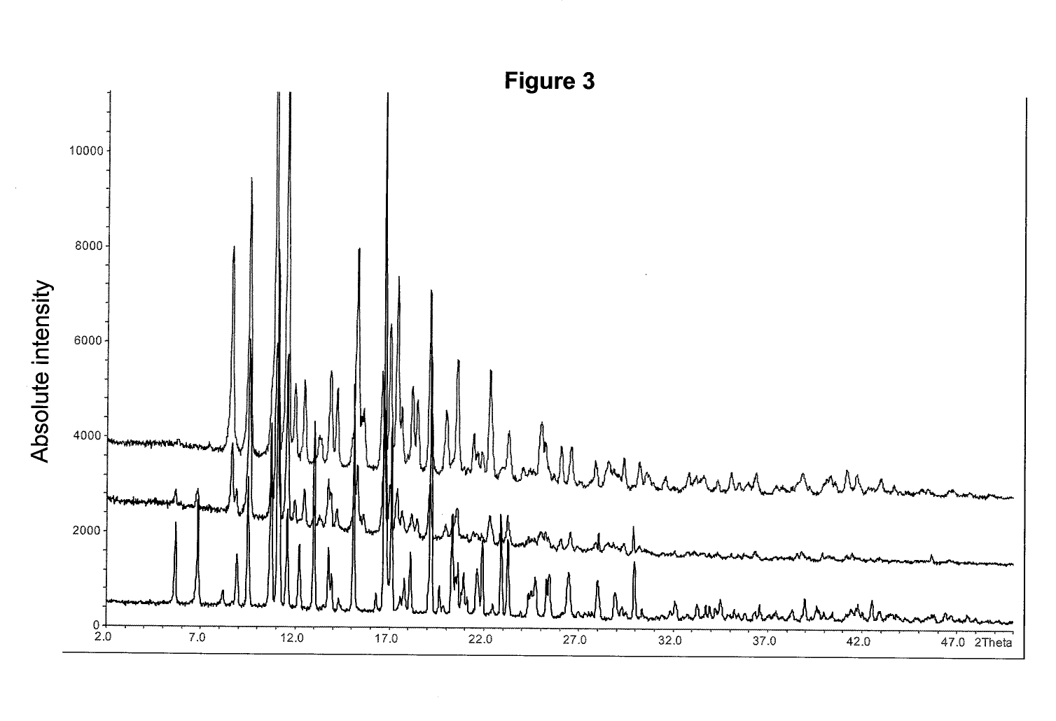

### DETAILED DESCRIPTION

Preferably the crystalline modification according to the invention contains substantially no organic solvent. For the purpose of the description, the expression “substantially no organic solvent” preferably means that the content of organic solvent is preferably at most 1000 ppm, more preferably at most 800 ppm, even more preferably at most 600 ppm, most preferably at most 400 ppm and in particular at most 200 ppm.

Preferably the crystalline modification according to the invention displays, in DSC investigations, an endothermic reaction with peak temperature at 106±5° C., more preferably 106±4° C., even more preferably 106±3° C., most preferably 106±2° C. and in particular 106±1° C. and/or an endothermic reaction with peak temperature 229±5° C., more preferably 229±4° C., even more preferably 229±3° C., most preferably 229±2° C. and in particular 229±1° C.

Preferably, in the temperature range from 20 to 40° C., the crystalline modification according to the invention is thermodynamically less stable than form II.

Dosage forms now being marketed usually contain clarithromycin in the thermodynamically stable form II. Usually, however, solubility decreases as thermodynamic stability increases. The reduced thermodynamic stability of form V according to the invention compared with form II is therefore usually accompanied by comparatively better solubility, which may improve bioavailability and therefore offers important advantages during formulation of the active substance.

Moreover, the ketone, which is preferably used in the production of form V according to the invention, preferably acetone, can be removed easily due to its comparatively low boiling point and furthermore appears not to form any solvates with clarithromycin.

Another aspect of the present invention relates to a method for the production of the crystalline modification of clarithromycin described above comprising the step of
(a) dissolution of clarithromycin in a ketone.

Preferably, in the method according to the invention the ketone is selected from the group consisting of acetone, butan-2-one, pentan-2-one, pentan-3-one, hexan-2-one, hexan-3-one and cyclohexanone. Acetone is particularly preferred.

Preferably, in the method according to the invention, clarithromycin of form I is dissolved in step (a), i.e. form I is used as starting material. However, it is also possible to use other forms as starting material, for example form II.

Preferably, in the method according to the invention, the concentration of dissolved clarithromycin in the ketone, preferably in acetone, is at most 0.10 g/ml, more preferably at most 0.08 g/ml, even more preferably at most 0.07 g/ml, most preferably at most 0.06 g/ml and in particular at most 0.05 g/ml.

Preferably, in the method according to the invention, step (a) is carried out at a temperature higher than 20° C., more preferably higher than 40° C., even more preferably higher than 60° C. and/or in particular under reflux.

Preferably, in the method according to the invention, the raised temperature is maintained for 10 to 60 minutes, more preferably 20 to 45 minutes and in particular for 25 to 35 minutes.

Particularly preferably, in the method according to the invention, reflux is maintained for 10 to 60 minutes, more preferably 20 to 45 minutes and in particular for 25 to 35 minutes.

Preferably the method according to the invention further comprises the step
(b) separation of crystallization nuclei of undesirable polymorphic forms from the solution obtained in step (a).

Suitable methods for separating crystallization nuclei of undesirable polymorphic forms are known to a person skilled in the art. Preferably, in the method according to the invention, step (b) is carried out by filtration of the solution. Preferably the filtration is carried out at a temperature that is 0 to 60° C. below, preferably 0 to 40° C. below, more preferably 0 to 20° C. below, even more preferably 0 to 10° C. below the boiling point of the solvent being used. Particularly preferably, the filtration is carried out at the boiling point of the solvent being used. Undesirable polymorphic forms are, in this context, preferably all polymorphic forms of clarithromycin that do not correspond to form V according to the invention.

Preferably the method according to the invention further comprises the step
(c) precipitation of clarithromycin from the solution obtained in step (a) or in step (b).

Suitable methods of precipitation are known to a person skilled in the art. Preferably, in the method according to the invention, step (c) is carried out by cooling the solution. Preferably it is cooled to a temperature of at most 25° C., more preferably at most 20° C., even more preferably at most 10° C. and in particular at most 0° C.

Preferably, in the method according to the invention, after the precipitation in step (c), all other partial steps of step (c) take place at a temperature of at most 25° C., more preferably at most 20° C., even more preferably at most 10° C. and in particular at most 0° C.

Preferably the method according to the invention further comprises the step (d) drying of the precipitate obtained in step (c).

Preferably, in the method according to the invention, step (d) takes place under vacuum, more preferably at a vacuum of 1.0 to 900 mbar, even more preferably at a vacuum of 10 to 500 mbar, and in particular at a vacuum of 20 to 200 mbar.

Preferably, in the method according to the invention, step (d) takes place in a temperature range from 0 to 90° C., more preferably from 10 to 70° C., even more preferably from 20 to 50° C.

Preferably, in the method according to the invention, step (d) takes place for 5 to 60 minutes, more preferably for 10 to 50, even more preferably 20 to 40 minutes.

Another aspect of the present invention relates to a crystalline modification of clarithromycin that can be obtained by the method described above.

Yet another aspect of the invention relates to the use of the crystalline modification of clarithromycin described above for the production of a medicament for the treatment and/or prevention of a bacterial infection, and to a method of treating a bacterial infection comprising administering an effective anti-bacterial amount of crystalline clarithromycin (form V).

Preferably the bacterial infection is selected from the group consisting of bacterial infections of the respiratory tract, infections of the upper and lower respiratory tract, bacteria-induced bronchitis, chronic bronchitis, pneumonia, atypical pneumonia, pharyngitis, sinusitis, tonsillitis and otitis media.

The following examples are intended to provide a more detailed explanation of the invention, but are not to be regarded as limiting.

### EXAMPLE 1

### Synthesis of the Pure Form V

1.06 g of clarithromycin form I was weighed in a 50-ml single-neck flask and then suspended in 13 ml acetone p.a. The suspension was stirred with a magnetic stirrer (Heidolph MR 3003) at stage 4, heating on an oil bath to 75° C. to reflux. As the suspension did not immediately dissolve completely even under these conditions, a further 10 ml acetone was added, producing a solution. The mixture was heated for 30 minutes to reflux and, while still hot, was filtered with suction through a G4 filter frit, applying a light vacuum, in order to remove solid particles. After cooling to room temperature, the Erlenmeyer flask was cooled for approx. 0.5 h on an ice bath. Then solid matter adhering to the flask was scraped away with a spatula and was well-filtered by suction with the mother liquor through a filter frit G4. The solid thus obtained was dried in a drying cabinet at 45° C. and 100 mbar pressure for 45 min.

The yield of crystalline solid was 0.602 g (56.8% of theoretical)

### EXAMPLE 2

### Synthesis of Form V Mixed with Form II

The same form V was also observed on recrystallization of form II from acetone. In the powder diffraction pattern, in addition to reflections of form II, those of form V are also clearly visible.

For this purpose, 3.0 g clarithromycin form II was weighed in a 50-ml single-neck flask, suspended in 30 ml acetone p.a. and stirred at stage 4 by means of a magnetic stirrer bar (Heidolph MR 3003). The suspension was heated on an oil bath to 75° C. to reflux. As the weighed portion of solid had still not dissolved completely, a further 10 ml acetone was added. The mixture was heated for 30 minutes to reflux and was filtered off while still hot on a pleated filter (Schleicher&Schüll Micro Science, diameter 185, Ref. No. 10311647). After cooling to room temperature, the filtrate was cooled for approx. 1 hour on an ice bath. The resulting solid was put on a filter frit D3 and filtered with suction. Then air was sucked through for approx. 3 min more. The yield was 1.967 g (65.6% of theoretical)

### XRPD Analysis

### X-Ray Powder Diffractometry (XRPD, X-Ray Powder Diffraction):

XRPD investigations were conducted with a STOE Stadi P X-ray powder diffractometer in transmission geometry, using CuKα₁ radiation made monochromatic by means of a germanium single crystal. D-spacings are calculated from the 20 values, based on a wavelength of 1.54060 Å. As a general rule the 20 values have an error rate of ±0.2° in 2Θ. The experimental error for the d-spacing values therefore depends on the position of the line (of the peak).

Table 1 shows the list of peaks for form V. The uncertainty in the 2Θ values is ±0.2 in 2Θ. rel. I shows the relative intensity of the particular peak. Maximum intensity is 100. The evaluation of the peaks relates to patent WO 01/44262 for form IV (p. 3 ff.).

As can be seen from the detailed comparisons in the following tables, form V is a form that has not been described previously:

TABLE 1

| 2Θ | rel. I |
|---|---|
| 5.64 | 19 |
| 6.84 | 23 |
| 8.18 | 7 |
| 8.92 | 13 |
| 9.49 | 27 |
| 10.73 | 39 |
| 11.08 | 70 |
| 11.58 | 22 |
| 12.21 | 15 |
| 12.99 | 38 |
| 13.76 | 15 |
| 13.92 | 9 |
| 14.29 | 5 |
| 15.09 | 45 |
| 16.27 | 6 |
| 16.73 | 100 |
| 17.09 | 38 |
| 17.55 | 6 |
| 17.77 | 9 |
| 18.09 | 14 |
| 19.18 | 52 |
| 19.63 | 8 |
| 19.84 | 4 |
| 20.47 | 9 |
| 20.60 | 12 |
| 20.91 | 10 |
| 21.08 | 6 |
| 21.62 | 11 |
| 21.90 | 16 |
| 22.44 | 4 |
| 22.88 | 21 |
| 23.25 | 16 |
| 23.92 | 3 |

The lattice constants were determined by using the VISSER program: (VISSER program: implemented e.g. in the WinXPow software (Version 2.11 or 2.15) from the company STOE.) Table 2 gives the lattice constants of form V (determined with VISSER, the value for the b-axis was halved).

TABLE 2

| a/Å | b/2/Å | c/Å | α/° | β/° | γ/° | Cell volume/Å³ |
|---|---|---|---|---|---|---|
| 10.031 | 26.141 | 8.788 | 90 | 90 | 90 | 4109 |

Comparison of the lattice constants of form V with other, known forms.

| | |
|---|---|
| Form V | 4109 Å³ |
| WANNUU MeOH solvate | 4335 Å³ |
| NAVSUY Stephenson et al. 1997 | 4267 Å³ |
| Form III acetonitrile solvate | 2196 Å³ |

The cell volume of form V according to the invention is therefore smaller than the cell volume of the WANNU MeOH solvate and of the NAVSUY form, which, if we assume an ansolvate for form V, means a higher density determined radiographically.

Comparison of the Powder Diffraction Pattern of Form V with the Other Known Forms

Table 3 only lists, in each case, the X-ray diffraction reflections for which there are no corresponding reflections of the other form considered in each case within ±0.2 2Θ (“solo characteristics”):

TABLE 3

| 2Θ | | | | | | | rel. I |
|---|---|---|---|---|---|---|---|
| | | | Form | | Form | Form | |
| NAV-SUY | Form 0 | Form I | II | Form III | IV | V | |
| | 4.70 | | | | | — | — |
| | | 5.16 | | | | — | — |
| | — | — | — | — | — | 5.64 | 19 |
| — | | | — | — | — | 6.84 | 23 |
| | | | | | 7.60 | — | — |
| | 7.70 | | | | | — | — |
| 7.38 | | | | | | — | — |
| — | — | — | | — | | 8.18 | 7 |
| | | — | | | — | 8.92 | 13 |
| | | — | | | | 9.49 | 27 |
| | | 10.20 | | | | — | — |
| | | — | | — | | 10.73 | 39 |
| | | — | | — | | 11.08 | 70 |
| | | — | | | — | 11.58 | 22 |
| | | | | — | | 12.21 | 15 |
| | | — | — | | — | 12.99 | 38 |
| | | — | | — | — | 13.76 | 15 |
| | | | | — | — | 13.92 | 9 |
| | | | | — | — | 14.29 | 5 |
| | | 15.72 | | | | — | — |
| | — | | | | | 16.27 | 6 |
| | — | | | | | 16.73 | 100 |
| | | — | | | — | 17.09 | 38 |
| | | | | — | — | 17.55 | 6 |
| | — | | | — | — | 17.77 | 9 |
| | — | | | | — | 18.09 | 14 |
| | 18.50 | | | | | — | — |
| | | | | — | | 19.63 | 8 |
| | | | | | 18.70 | — | — |
| | | | | — | — | 19.84 | 4 |
| | — | | | — | | 20.31 | 21 |
| | — | | | — | | 20.47 | 9 |
| | — | | | — | | 20.60 | 12 |
| | — | | — | — | | 20.91 | 10 |
| | — | | | — | | 21.08 | 6 |
| | — | | | — | | 21.62 | 11 |
| | — | | | — | | 21.90 | 16 |
| | — | | | — | | 22.44 | 4 |
| | | | | — | | 22.88 | 21 |
| | | | | — | | 23.25 | 16 |
| | | | | — | | 23.92 | 3 |
| | | | | — | | 24.34 | 7 |
| | — | | | — | | 24.47 | 7 |
| | | | | — | | 25.43 | 10 |

— diffraction reflection not present within ±0.2 2Θ

### DSC Analysis

Differential Scanning Calorimetry (DSC): equipment designation Mettler Toledo DSC821e. Unless indicated otherwise, the samples were weighed in aluminium crucibles (volume=40 μL) and covered with a perforated lid. Measurement was carried out with nitrogen flow rate of 50 mL/min in the temperature range from 30 to 260° C. with a heating rate of 10 K/min. Unless stated otherwise, the temperatures quoted in connection with DSC investigations are the temperatures of the peak maxima (peak temperature T_{P}). T_{O} denotes onset temperatures of peaks.

### DSC

T_{O} 98.57; Tₚ 106.33; J/g 34.10;
T_{O} 228.20; Tₚ 228.76; J/g 65.55

In thermal analysis (DSC), form V shows endothermic reactions with peak temperatures at 106° C. and at 229° C. The first endothermic event indicates a phase transformation. The second endothermic reaction is higher than that described in the literature for form I and form II.

Comparison of DSC events described in the literature (peak temperatures are given in degrees Celsius)—Table 4:

TABLE 4

| Events | Events | Events | Events | Events |
|---|---|---|---|---|
| Form V | Form 0 | Form I | Form II | Form IV |
| Endo 106.3 | | | | |
| | | Endo 132.2 | | |
| Endo 228.8 | | Endo 223.4 | Endo 223.4 | |
| | | Endo 283.3 | Endo 283.3 | |
| | | Exo 306.9 | | |

However, DSC thermograms of forms I and II, which were recorded under the same conditions as those of form V (cf. appendix, equipment and methods), show that the endothermic event with peak temperature at 229±1° C. occurs both on investigation of forms I and II and on investigation of form V (Endo denotes endothermic event, temperatures in ° C.)—Table 5:

TABLE 5

| Events | Events | Events | Events | Events |
|---|---|---|---|---|
| Form V | Form 0 | Form I | Form II | Form IV |
| Endo 106.3 | | | | |
| | | Endo 147.7 | | |
| Endo 228.8 | | Endo 228.4 | Endo 228.4 | |

### Thermodynamic Stability

To investigate physical stability in comparison with the known forms form I and form II, the following experiments were carried out (interconversion tests):
a) about 100 mg of form I and approx. 100 mg of form II were put in a 20 ml vial and suspended in 2.5 ml acetone p.a.
b) about 100 mg of form V and approx. 100 mg of form I were put in a 20 ml vial and suspended in 2.5 ml acetone p.a.
c) about 100 mg of form V and approx. 100 mg of form II were put in a 20 ml vial and suspended in 2.5 ml acetone p.a.

All three suspensions were first shaken at room temperature (approx. 20° C.) on a PLS synthesizer at 400 rpm for approx. 2 h. For 20 h the temperature of the suspensions was raised to 40° C. and then left to cool to room temperature (approx. 20° C.) again. The solids did not dissolve completely even at 40° C., so that suspension was present for the whole duration of the test.

Then the solids were filtered with suction on a G4 frit, made into a slurry with 0.5 ml acetone p.a., washed, and then dried by suction in an air stream, applying a light vacuum.

The crystalline solids were in each case characterized by DSC, XRPD, Raman and TG investigations. The results of the interconversion tests in acetone are presented in Table 6:

TABLE 6

| Experiment | Starting mixture | Product |
|---|---|---|
| a) | Form I/Form II | Form II |
| b) | Form V/Form I | Form II |
| c) | Form V/Form II | Form II |

Accordingly, under the test conditions, form II is both thermodynamically more stable than form V as well as more stable than form I. The latter is in agreement with the findings reported in the literature.

The foregoing description and examples have been set forth merely to illustrate the invention and are not intended to be limiting since modifications of the disclosed embodiments incorporating the spirit and substance of the invention may occur to persons skilled in the art, the invention should be construed broadly to include all variations within the scope of the appended claims and equivalents thereof.

What is claimed is:

1. A crystalline modification of clarithromycin which exhibits X-ray diffraction reflections at 5.64±0.20 2Θ, 6.84±0.20 2Θ, 8.92±0.20 2Θ, 11.58±0.20 2Θ, 12.99±0.20 2Θ and 16.73±0.20 2Θ.

2. A crystalline modification according to claim 1, wherein said crystalline modification additionally exhibits at least one X-ray diffraction reflection selected from the group consisting of 8.18±0.20 2Θ, 9.49±0.20 2Θ, 10.73±0.20 2Θ, 11.08±0.20 2Θ, 12.21±0.20 2Θ, 13.76±0.20 2Θ, 13.92±0.20 2Θ, 17.09±0.20 2Θ, 17.77±0.20 2Θ, 18.09±0.20 2Θ, 19.18±0.20 2Θ, 20.31±0.20 2Θ, 20.60±0.20 2Θ and 20.91±0.20 2Θ.

3. A crystalline modification according to claim 1, wherein said crystalline modification is substantially free of organic solvent.

4. A crystalline modification according to claim 1, wherein in Differential Scanning Calorimetry said crystalline modification exhibits an endothermic reaction at 106±5° C. or at 229±5° C. or endothermic reactions at both 106±5° C. and 229±5° C.

5. A crystalline modification according to claim 1, wherein in the temperature range from 20 to 40° C., said crystalline form is thermodynamically less stable than form II.

6. A method of producing a crystalline modification of clarithromycin which exhibits X-ray diffraction reflections at 5.64±0.20 2Θ, 6.84±0.20 2Θ, 8.92±0.20 2Θ, 11.58±0.20 2Θ, 12.99±0.20 2Θ and 16.73±0.20 2Θ, said method comprising:
(a) dissolving clarithromycin in a ketone to obtain a solution, and
(b) recovering said crystalline modification from said solution.

7. A method according to claim 6, wherein said ketone is acetone.

8. A method according to claim 6, wherein clarithromycin of form I is dissolved in step (a).

9. A method according to claim 6, wherein the concentration of dissolved clarithromycin in the ketone is at most 0.10 g/ml.

10. A method according to claim 6, wherein the dissolving is carried out under reflux.

11. A method according to claim 10, wherein the reflux is maintained for 10 to 60 minutes.

12. A method according to claim 6, further comprising separating crystallization nuclei of undesirable polymorphic forms from the solution obtained in step (a).

13. A method according to claim 6, wherein the recovering comprises precipitating clarithromycin from the solution obtained in step (a).

14. A method according to claim 13, wherein the precipitation is carried out by cooling the solution.

15. A method according to claim 13, further comprising drying the precipitate obtained in the precipitating step.

16. A method according to claim 15, wherein the drying is carried out under vacuum.

17. A method according to claim 16, wherein the vacuum is in the range from 10 to 500 mbar.

18. A method according to claim 15, wherein the drying is carried out for 10 to 60 minutes.

19. A method according to claim 15, wherein the drying is carried out at a temperature from 10° C. to 70° C.

20. A crystalline modification of clarithromycin obtained by the method according to claim 6.

21. A method of treating a bacterial infection in a subject, said method comprising administering to said subject an effective anti-bacterial amount of a crystalline modification of clarithromycin which exhibits X-ray diffraction reflections at 5.64±0.20 20, 6.84±0.20 2Θ, 8.92±0.20 2Θ, 11.58±0.20 2Θ, 12.99±0.20 2Θ and 16.73±0.20 2Θ.

22. A method according to claim 21, wherein said bacterial infection is selected from the group consisting of bacterial infections of the respiratory tract, bacterial infections of the upper and lower respiratory tract, bacteria-induced bronchitis, chronic bronchitis, pneumonia, atypical pneumonia, pharyngitis, sinusitis, tonsillitis and otitis media.
